(19) United States
(12) Patent Application Publication
(10) Pub. No.: US 2003/0219478 A1
(43) Pub. Date: Nov. 27, 2003

(54) Capsule preparation

(76) Inventors: Hiroshi Ohnuki, Tokyo (JP); Takahisa Takubo, Tokyo (JP); Eisaku Sai, Tokyo (JP); Kenji Miyata, Tokyo (JP)

Correspondence Address: Evan J. Federman, Legal Division, Warner-Lambert Company LLC, 201 Tabor Road, Morris Plains, NJ, 07950, US

(21) Appl. No.: 10/408,536

(22) Filed: Apr. 7, 2003

(30) Foreign Application Priority Data
Apr. 24, 2002 (JP) 2002-121941

(51) Int. Cl. A61K 31/70; A61K 31/573; A61K 31/51; A61K 31/375; A61K 31/11; A61K 9/48

(52) U.S. Cl. 424/452; 514/23; 514/37; 514/474; 514/179; 514/693; 514/699; 514/276

(57) ABSTRACT

A capsule is made by filling a medical formulation, food or food additive which has an aldehyde group in the molecule, or readily forms an aldehyde group, into a capsule made of pullulan as a base.

This application claims priority to Japanese Patent Application 2002-121941, filed Apr. 24, 2002.

The present invention relates to a novel capsule preparation and, specifically, to a capsule preparation with stable appearance quality into which vitamin C or the like is filled.

### BACKGROUND OF THE INVENTION

Capsule preparations prepared by filling a drug into a gelatin capsule are known. However, capsule preparations prepared by filling vitamin C or the like into a gelatin capsule have a problem that the capsule turns brown with the passage of time, exerting a fatal influence upon the appearance quality.

Therefore, means of retaining good appearance quality by preventing or suppressing the discoloration of the capsule preparations into which a drug such as vitamin C or food is filled has been desired.

### DETAILED DESCRIPTION OF THE INVENTION

The inventors of the present invention have conducted intensive studies to solve the above problem and have accomplished the present invention.

The present invention relates to a capsule preparation prepared by filling a medical formulation, food or food additive which has an aldehyde group in the molecule, or readily forms an aldehyde group, into a capsule made of pullulan as a base.

Pullulan is a linear α-glucan wherein α-1,4 bonded maltotrioses are repeatedly polymerized at both terminals through α-1,6-bond and it is a water-soluble polysaccharide produced outside the body by Aureobasidium pullulans.

The capsule made of pullulan as a base can be produced by a known method. For example, as described in WO/01/07507 (PTC/EP00/06843), it can be preferably produced by an immersion molding method in which a capsule is formed from a film forming composition containing pullulan and a cured system using a capsule production machine which is commonly used for the production of a hard gelatin capsule.

### BRIEF DESCRIPTION OF THE DRAWING

FIG. 1 The photos show the inspection result of the capsule made of gelatin as a base (left photo) of the capsule preparation and the capsule made of pullulan as a base (right photo) of the capsule preparation, after six weeks of preservation.

A large number of medical formulations, foods or food additives which have an aldehyde group in the molecule, or readily form an aldehyde group, are present and all of them can be used in the present invention. The following are exemplified: monosaccharides and derivatives thereof such as erythrose, threose, ribose, arabinose, xylose, lyxose, allose, glucose, mannose, gulose, idose, galactose and talose; food additives such as anisaldehyde, L-perillaldehyde, α-amylcinnamaldehyde, cinnamaldehyde, benzaldehyde, decanal, octanal, vanillin, ethyl vanillin, citral, citronellal, hydroxycitronellal, dibenzoylthiamin, dibenzoylthiamin hydrochloride, bisbentiamine and piperonal; medical formulations such as vitamin C, fursultiamine hydrochloride (vitamin B1), aminoglucoside-type antibiotics such as streptomycin sulfate, macrolide-type antibiotics such as rokitamycin, acetylspiramycin, leucomycin and midecamycin acetate, and betamethasone valerate. Among them, monosaccharides and derivatives thereof such as erythrose, threose, ribose, arabinose, xylose, lyxose, allose, glucose, mannose, gulose, idose, galactose and talose, vitamin C and fursultiamine hydrochloride (vitamin B1) are preferred.

Although the capsule preparation of the present invention contains a medical formulation, food or food additive which has an aldehyde group in the molecule, or readily forms an aldehyde group, such as vitamin C, the discoloration of the capsule with the passage of time is prevented or suppressed and its good appearance quality is retained.

The mechanism that the discoloration is prevented or suppressed by the present invention is unknown but it is considered that the Maillard reaction between the capsule base material and a formulation, food or food additive such as vitamin C is suppressed.

### EXAMPLES

The following examples are provided for the purpose of further illustrating the present invention but the invention is not limited by the examples.

### Example 1

### Production of Capsule Preparation

A capsule which was made of pullulan as a base and colored white with titanium oxide was produced in the same manner as in Example of WO/01/07507. That is, 20 g of hydrolyzed, deoiled lecithin, 363 g of K-carrageenan and 40 kg of pullulan were dispersed into 142 liters of deionized water by heating at 70° C. with agitation. An aqueous solution containing 455 g of potassium acetate was added to this mixture. 3 liters of the thus prepared pullulan solution, 3 liters of hot water and 800 g of titanium dioxide were mixed together and defoamed to prepare a slurry solution at 60° C. A white, opaque hard pullulan capsule was produced from this slurry in the same manner as a hard pullulan capsule was.

350 mg of powdered vitamin C were filled into each capsule to prepare a capsule preparation.

For comparison, a commonly used capsule made of gelatin as a base was prepared and filled with powdered vitamin C in similar manner to the above.

### Example 2

### Preservation Test

The pullulan capsule preparation obtained in Example 1 and the gelatin capsule preparation for comparison were placed in a high-density polyethylene container with a cover and preserved under an environment of 40° C. and a humidity of 75% for 6 weeks. After preservation, the appearance quality of the capsules in the capsule preparations was inspected with the naked eye.

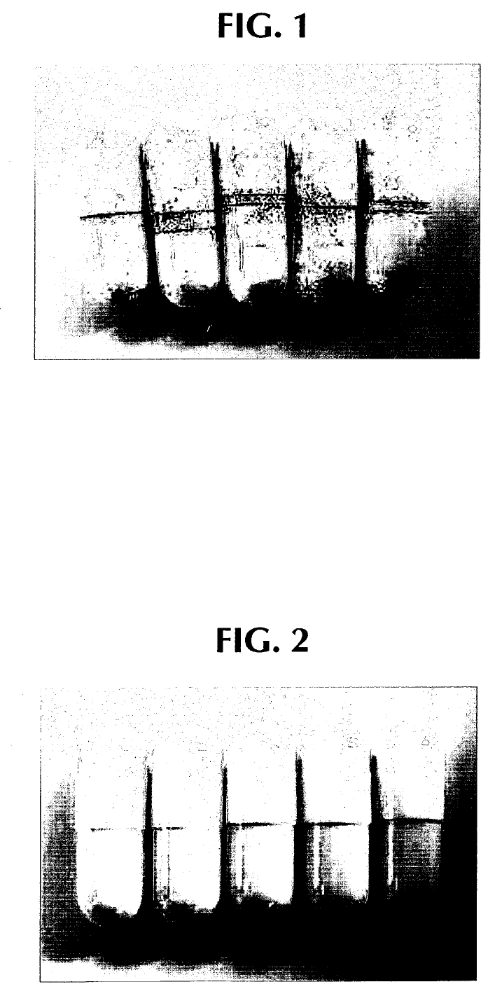

As shown in FIG. 1, the inspection revealed that after six weeks of preservation, the capsule made of gelatin as a base (left photo) turned brown whereas the discoloration of the capsule made of pullulan as a base (right photo) was not seen.

The capsule preparation of the present invention can retain its good appearance quality because the discoloration of the capsule with the passage of time is prevented or suppressed.

We claim:

1 A capsule preparation comprising a filling having a composition including an aldehyde group in the molecule, or a composition which readily forms an aldehyde group, wherein said capsule comprises pullulan.

2 The capsule preparation of claim 1, wherein said composition including an aldehyde group in the molecule, or said composition which readily forms an aldehyde group, is selected from the group consisting of erythrose, threose, ribose, arabinose, xylose, lyxose, allose, glucose, mannose, gulose, idose, galactose, talose, anisaldehyde, L-perillaldehyde, α-amylcinnamaldehyde, cinnamaldehyde, benzaldehyde, decanal, octanal, vanillin, ethyl vanillin, citral, citronellal, hydroxycitronellal, dibenzoylthiamin, dibenzoylthiamin hydrochloride, bisbentiamine, piperonal, vitamin C, fursultiamine hydrochloride, streptomycin sulfate, rokitamycin, acetylspiramycin, leucomycin, midecamycin acetate and betamethasone valerate.

3 The capsule preparation of claim 2, wherein said composition including an aldehyde group in the molecule, or said composition which readily forms an aldehyde group, is selected from the group consisting of erythrose, threose, ribose, arabinose, xylose, lyxose, allose, glucose, mannose, gulose, idose, galactose, talose, vitamin C and fursultiamine hydrochloride.

4 The capsule preparation of claim 3, wherein said composition including an aldehyde group in the molecule, or said composition which readily forms an aldehyde group, comprises vitamin C.
